(19) United States
(12) Patent Application Publication
(10) Pub. No.: US 2010/0268033 A1
(43) Pub. Date: Oct. 21, 2010

(54) CAPSULE ENDOSCOPE

(76) Inventor: Chikara Yamamoto, Saitama (JP)

Correspondence Address: BIRCH STEWART KOLASCH & BIRCH, PO BOX 747, FALLS CHURCH, VA, 22040-0747, US

(21) Appl. No.: 12/761,974

(22) Filed: Apr. 16, 2010

(30) Foreign Application Priority Data
Apr. 17, 2009 (JP) 2009-100650

(51) Int. Cl. A61B 1/06

(52) U.S. Cl. 600/178

(57) ABSTRACT

A capsule endoscope includes a capsule body having a cylindrical section, and a transparent cover. In the capsule endoscope, a reflective optical system, an image forming optical system, LEDs, and an image sensor are contained. The cylindrical transparent cover is disposed in the middle of the cylindrical section in a longitudinal direction. Through the transparent cover, light is transmittable in 360 degrees around a central axis of the capsule body. The reflective optical system reflects the light that has entered the capsule body through the transparent cover to the image forming optical system. The image forming optical system forms a 360 degree image around the whole circumference of the cylindrical section on the image sensor. The 360 degree image is circular in shape.

### BACKGROUND OF THE INVENTION

1. Field of the Invention

The present invention relates to a swallowable capsule endoscope for imaging the inside of digestive organs.

2. Description Related to the Prior Art

In medical endoscopy, a wireless capsule endoscope in which an image sensor is provided in a small capsule is now common, in addition to a flexible tube type wired endoscope whose slender insert section is introduced into patient's mouth and image sensor provided at a distal end of the insert section images the inside of a body. In the capsule endoscopy, the image sensor provided in the capsule endoscope images the inside of digestive organs at predetermined time intervals, while the capsule swallowed by the patient is moving along the digestive system. Endoscopy with use of the flexible tube type endoscope often imposes a heavy burden on the patient, because the patient has to put up with insertion of the tube (insert section) into his/her mouth throughout examination. The capsule endoscope, on the other hand, can reduce the burden on the patient, because all the patient has to do is to swallow the capsule having the image sensor.

In the capsule endoscope, an imaging lens, the image sensor, and a light source are contained in an approximately cylindrical hollow capsule body. To an end of the capsule body, a dome-shaped transparent cover is attached for covering the imaging lens. Light emitted from the light source is applied to an internal body part through the transparent cover. The image sensor captures an image of the illuminated body part through the transparent cover and the imaging lens.

Since the position of the capsule endoscope is hard to control inside the body, it is difficult to keep an imaging area constant. Thus, the capsule endoscope adopts a wide-angle imaging lens so as to image a wide area inside the body. The wide-angle imaging lens ensures imaging of the area in front of the dome-shaped transparent cover of the capsule body. However, imaging of the other directions, e.g. side directions of the capsule body is still difficult even with further widening of the angle of the imaging lens. Accordingly, a lesion in the side direction of the capsule body cannot be imaged, and is overlooked at diagnosis.

According to a capsule endoscope of Japanese Patent Laid-Open Publication No. 2007-159642, on the other hand, a cylindrical transparent cover is attached to the middle of a capsule body in the longitudinal direction. An imaging lens and an image sensor are rotated along the inner circumference of the transparent cover, so as to image areas around the whole circumference of the capsule body.

The above capsule endoscope, however, requires a drive unit for rotating the imaging lens and the image sensor, and becomes large in size. Also, a high-power battery, which is required for feeding electric power to not only the image sensor but also the drive unit, causes further upsizing and increase in cost.

### SUMMARY OF THE INVENTION

An object of the present invention is to provide a capsule endoscope that can image an area along a cylindrical section of a capsule body at low cost and in small size.

A capsule endoscope according to the present invention is constituted of a capsule body having a cylindrical section, a cylindrical transparent cover, a reflective optical system, an image forming optical system, and an image sensor. The cylindrical transparent cover is attached to substantially a middle of the cylindrical section. Through the transparent cover, light is transmittable in 360 degrees around a central axis of the capsule body. The reflective optical system reflects light incident from the outside of the whole circumference of the cylindrical section. The image forming optical system forms an image from reflected light from the reflective optical system. The image sensor receives light of the image formed by the image forming optical system, and captures a 360 degree image around the cylindrical section at a time.

It is preferable that each of the reflective optical system and the image forming optical system be symmetric with respect to its axis. It is preferable that the reflective optical system be in the shape of a cone, and an outer conical surface of the cone reflect the light that has entered the capsule body through the transparent cover. It is also preferable that an optical axis of the image forming optical system coincide with a center line of the cone passing through an apex of the cone, and the center line of the cone extend in a longitudinal direction of the cylindrical section. The conical surface may have refractive power.

It is preferable that the image sensor be disposed orthogonally to the optical axis of the image forming optical system. A plane of focus of the image forming optical system is conical in shape, so that the distance between the plane of focus and the image forming optical system is longest in the vicinity of the optical axis of the image forming optical system, and is reduced with increase in a distance from the optical axis.

The capsule endoscope may further include a lighting section for applying emission light through the transparent cover to the outside of the capsule body. The lighting section is disposed in such a position that the emission light reflected from the transparent cover is not incident upon the image forming optical system.

The lighting section may have a ring-shaped light emitting unit, or a plurality of LEDs disposed at a predetermined interval. The lighting section may be disposed on the opposite side of the image forming optical system across the reflective optical system, and a space may be created between the reflective optical system and the transparent cover to pass the emission light from the lighting section therethrough.

The capsule endoscope may further have a first battery for feeding electric power to the image sensor and a second battery for feeding electric power to the lighting section. The first battery is disposed on the same side with the image sensor, and the second battery is disposed on the same side with the lighting section.

According to the present invention, an area outside of the whole circumference of the cylindrical section of the capsule body is imaged without rotating the image forming optical system and the image sensor. As a result, with the inexpensive and small capsule endoscope, it is possible to capture a 360 degree image that visualizes the outside of the whole circumference of the cylindrical section.

### BRIEF DESCRIPTION OF THE DRAWINGS

For more complete understanding of the present invention, and the advantage thereof, reference is now made to the following descriptions taken in conjunction with the accompanying drawings, in which:

FIG. 1 is a schematic view of a capsule endoscope system according to a first embodiment;

FIG. 2 is a longitudinal sectional view of a capsule endoscope according to the first embodiment;

FIG. 3 is a cross sectional view of the capsule endoscope;

FIG. 4 is a longitudinal sectional view of a capsule endoscope according to a second embodiment;

FIG. 5 is a cross sectional view of the capsule endoscope; and

FIG. 6 is a longitudinal sectional view of a capsule endoscope according to a third embodiment.

### DESCRIPTION OF THE PREFERRED EMBODIMENTS

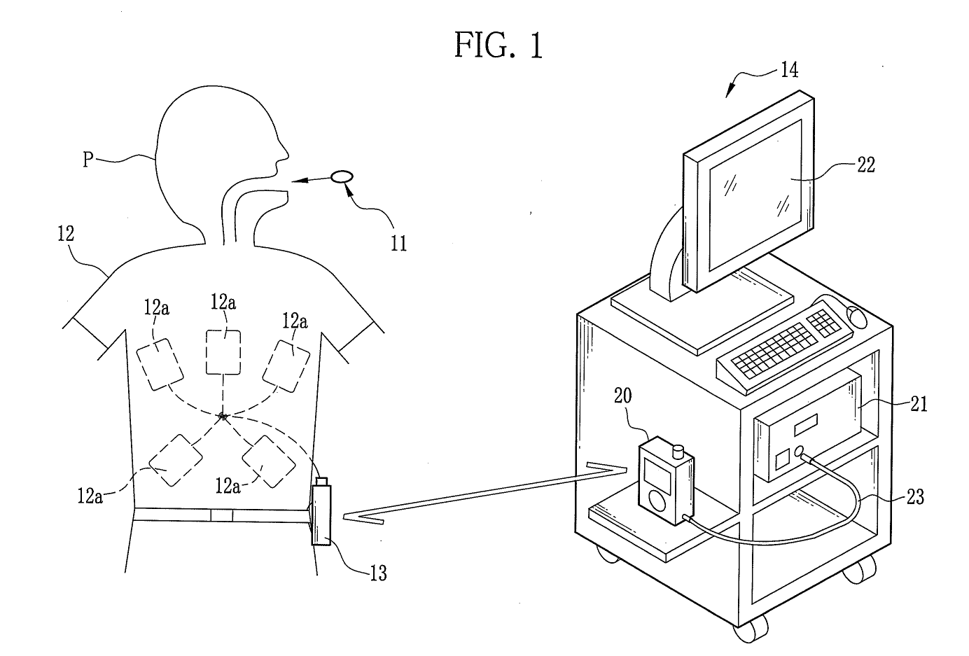

As shown in FIG. 1, a capsule endoscope system is constituted of a capsule endoscope 11, a shield shirt 12, a portable image storage device 13, and a work station 14. A patient P puts on the shield shirt 12 and the portable image storage device 13 before swallowing the capsule endoscope 11.

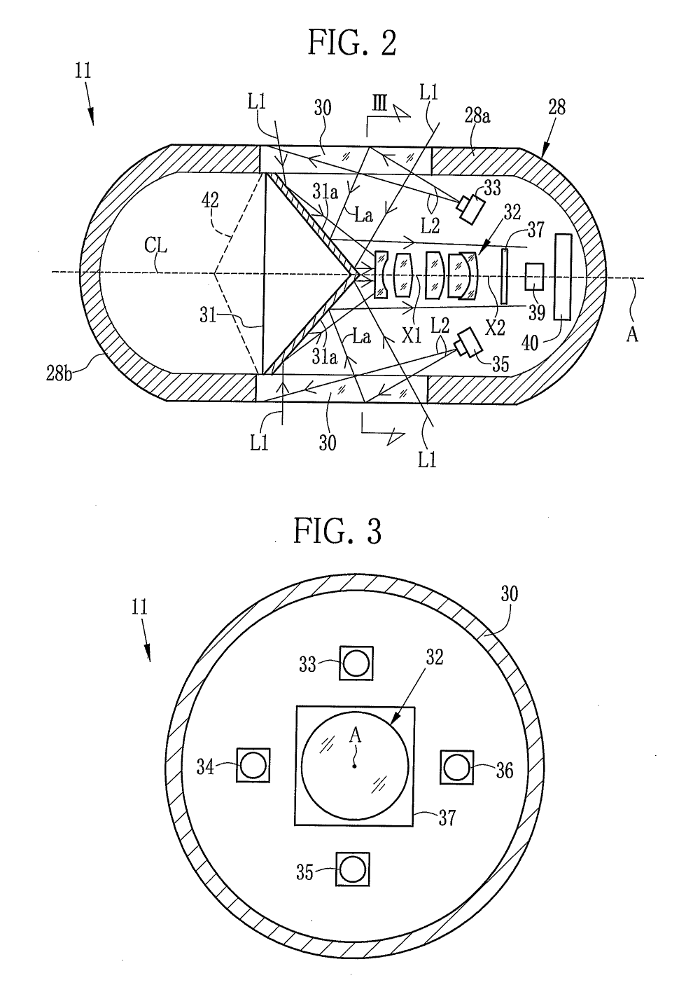

The capsule endoscope 11 is in an easily swallowable size, and captures images inside patient P's body during the period from being swallowed until excretion from the body. The captured images are sent to the outside at constant time intervals through an antenna 39 (refer to FIG. 2) provided in the capsule endoscope 11. The shield shirt 12 is provided with a plurality of signal receivers 12a for receiving an image signal from the capsule endoscope 11. The shield shirt 12 is made of a material that blocks electromagnetic waves and the like, so that signals other than the image signal, e.g. a signal from a cellular phone does not get in the signal receivers 12a. The portable image storage device 13 is connected to the signal receivers 12a, and stores the image signal, received by the signal receivers 12a, as image data. The portable image storage device 13 also wirelessly transmits the image data to an image receiving unit 20 of the work station 14.

The work station 14 has the image receiving unit 20, a processor 21, and a monitor 22. The processor 21 obtains the image data from the image receiving unit 20 through a USB cable 23. The processor 21 applies various types of image processing to the obtained image data. Based on the processed image data, an image is displayed on the monitor 22.

As shown in FIGS. 2 and 3, the capsule endoscope 11 is provided with a capsule body 28, a transparent or transparent cover 30, a reflective optical system 31, an image forming optical system 32, LEDs (Light Emitting Diodes) 33 to 36, an image sensor 37, the antenna 39, and a battery 40. The capsule body 28 is constituted of a cylindrical section 28a positioned in the middle of the capsule body 28 along a longitudinal direction and dome-shaped end sections 28b disposed on both ends of the cylindrical section 28a. The cylindrical section 28a and one of the dome-shaped end sections 28b contain the reflective optical system 31, the image forming optical system 32, the LEDs 33 to 36, the image sensor 37, the antenna 39, and the battery 40. The battery 40 feeds electric power to the LEDs 33 to 36 and the image sensor 37.

The cylindrical transparent cover 30 is attached to the middle of the cylindrical section 28a in the longitudinal direction. Through the transparent cover 30, light is transmittable in all directions, that is, in 360 degrees around a central axis A of the capsule body 28. Thus, light is applied to the outside of a whole circumference of the cylindrical section 28a of the capsule body 28 through the transparent cover 30, and light from the outside of the whole circumference of the cylindrical section 28a enters the capsule body 28 through the transparent cover 30.

The reflective optical system 31 is in the shape of a cone, and a reflective surface 31a is formed on an outer conical surface. The center line CL of the reflective optical system 31 passes through the apex of the cone, and coincides with an optical axis X1 of the image forming optical system 32 and an optical axis X2 of the image sensor 37, and is positioned on the central axis A of the capsule body 28. The reflective surface 31a reflects light L1 that has entered the capsule body 28 through the transparent cover 30 to the image forming optical system 32. Accordingly, the light L1 that has entered the capsule body 28 through the transparent cover 30 is gathered to the image forming optical system 32 by the reflective optical system 31, without use of any device for rotating the image forming optical system 32 inside the cylindrical section 28a as described in prior art. The reflective optical system 31 is in the shape of a triangle in a cross section passing through the center line CL, and is hollow inside for the sake of weight reduction. The apex of the cone of the reflective optical system 31 is at any appropriate angle, as long as the reflective optical system 31 can gather the light that has entered the capsule body 28 through the whole circumference or outer cylindrical surface of the transparent cover 30, in other words, from all 360 degrees around the center line CL, to the image forming optical system 32. The reflective surface 31a may be formed into a curved surface, e.g. a concave surface to give refractive power for light refraction. In this case, the reflective surface 31a becomes a surface of revolution about the center line CL.

The image forming optical system 32 includes five lens groups. The optical axis X1 of the image forming optical system 32 is orthogonal to an imaging surface of the image sensor 37. The image forming optical system 32 forms an image from the reflected light L1 on the imaging surface of the image sensor 37. As shown in FIG. 2, a plane 42 of focus of the image forming optical system 32 is conical in shape in the direction opposite to the cone of the reflective optical system 31. The distance between the plane 42 of focus and the image forming optical system 32 is the longest in the vicinity of the optical axis X1, and is reduced with increase in distance from the optical axis X1. As a matter of course, the lens groups are symmetric with respect to their axes, and the reflective optical system 31 is symmetric with respect to its axis.

The image sensor 37 receives the light L1 from the image forming optical system 32 on the imaging surface, and photoelectrically converts the light L1 into the image signal. Therefore, a circular 360 degree image that visualizes the outside of the circumference of the cylindrical section 28 is captured at a time. The image signal, which is produced by photoelectric conversion, is transmitted via the antenna 39 to the outside of the capsule body 28. If an antireflective surface is provided in a part of the outer conical surface 31a around the apex of the cone of the reflective optical system 31, a doughnut-shaped image is captured.

The four LEDs 33 to 36 are disposed around the central axis A of the capsule body 28 at 90 degree intervals, and apply light L2 through the transparent cover 30 to an internal body part. The light L2 is emitted in synchronization with the timing of image capture by the image sensor 37. Each of the LEDs 33 to 36 is disposed in such a position that reflected light La of the emission light L2 reflected from the transparent cover 30 is not incident upon the image forming optical system 32. Thus, the occurrence of ghost and flare is prevented. The number of the LEDs may be other than four.

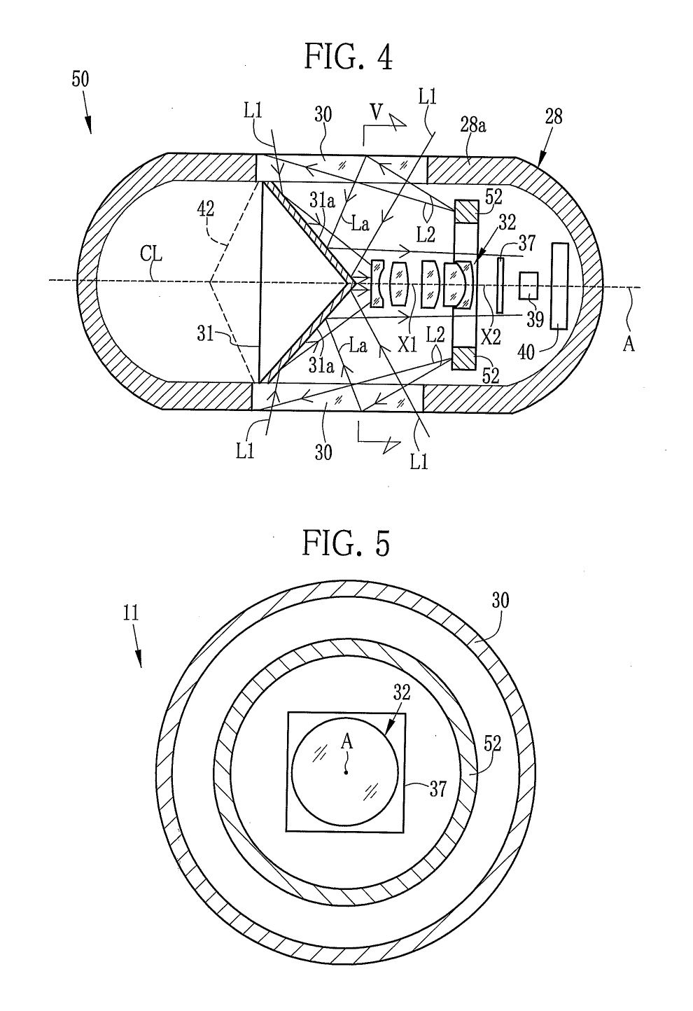

In a capsule endoscope 50 according to a second embodiment, as shown in FIGS. 4 and 5, a ring-shaped light emitting unit 52 is provided instead of the LEDs 33 to 36. The other components are identical to those of the capsule endoscope 11 of the first embodiment, and hence the description thereof will be omitted.

The ring-shaped light emitting unit 52 applies emission light L2 through the transparent cover 30 to an internal body part. The ring-shaped light emitting unit 52 is disposed in such a position that the center of the ring-shaped light emitting unit 52 is positioned on a central axis A of a capsule body 28, and reflected light La of the emission light L2 reflected from the transparent cover 30 is not incident upon an image forming optical system 32. Therefore, the occurrence of ghost and flare is prevented.

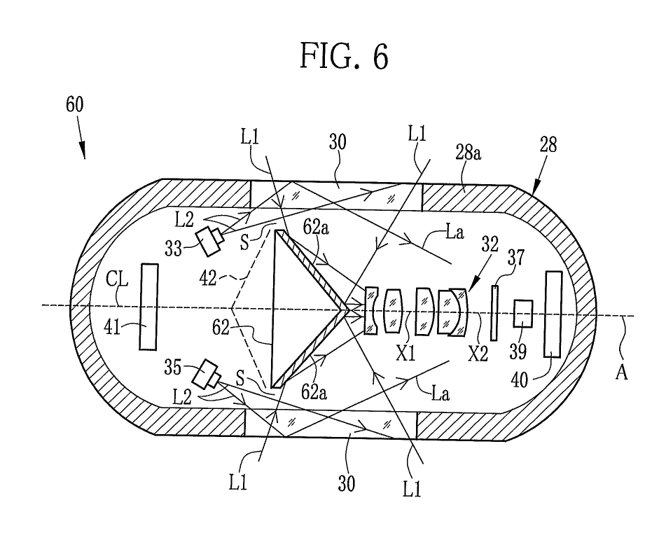

A capsule endoscope 60 according to a third embodiment, as shown in FIG. 6, has two batteries 40 and 41. The battery 40 is disposed on a right side of the capsule body 28, and the battery 41 is disposed on a left side of the capsule body 28. Thus, the batteries 40 and 41 disposed on both sides of the capsule body 28 maintain a weight balance of the capsule body 28. The battery 40 feeds electric power to the image sensor 37, and the battery 41 feeds electric power to the LEDs 33 to 36. Providing the two batteries 40 and 41 extends drive time of the image sensor 37 and the LEDs 33 to 36.

A conical reflective optical system 62 has a reflective surface 62a formed on an outer conical surface, as with the reflective surface 31a of the reflective optical system 31 according to the first embodiment. The reflective surface 62a reflects light L1 that has entered the capsule body 28 through a transparent cover 30. The rim of the reflective surface 62a does not reach the transparent cover 30, and a space S is created between the rim and the transparent cover 30 to pass the emission light L2 from the LEDs 33 to 36 therethrough.

The four LEDs 33 to 36 are disposed around a central axis A of the capsule body 28 at 90 degree intervals. The LEDs 33 to 36 are disposed on the opposite side of an image forming optical system 32 and an image sensor 37 across the reflective optical system 62. Accordingly, the light L2 emitted from the LEDs 33 to 36 passes through the space S and the transparent cover 30, and is applied to an internal body part. Since the light L2 is emitted from the opposite side of the image forming optical system 32 and the image sensor 37, as described above, even if the light L2 is reflected by the transparent cover 30, the reflected light La is not incident upon the image forming optical system 32. Therefore, the occurrence of ghost and flare is prevented.

The other components of the capsule endoscope 60 are identical to those of the capsule endoscope 11 according to the first embodiment, and hence the description thereof will be omitted.

Although the present invention has been fully described by the way of the preferred embodiment thereof with reference to the accompanying drawings, various changes and modifications will be apparent to those having skill in this field. Therefore, unless otherwise these changes and modifications depart from the scope of the present invention, they should be construed as included therein.

What is claimed is:

1. A capsule endoscope comprising:
a capsule body having a cylindrical section;
a cylindrical transparent cover attached to substantially a middle of the cylindrical section, for transmitting light in 360 degrees around a central axis of the capsule body;
a reflective optical system for reflecting light incident from an outside of a whole circumference of the cylindrical section through the transparent cover;
an image forming optical system for forming an image from reflected light from the reflective optical system; and
an image sensor for receiving light of the image formed by the image forming optical system, and capturing a 360 degree image around the cylindrical section at a time.

2. The capsule endoscope according to claim 1, wherein each of the reflective optical system and the image forming optical system is symmetric with respect to its axis.

3. The capsule endoscope according to claim 1, wherein the reflective optical system is in shape of a cone, and an outer conical surface of the cone reflects the light having entered the capsule body through the transparent cover.

4. The capsule endoscope according to claim 3, wherein an optical axis of the image forming optical system coincides with a center line of the cone passing through an apex of the cone, and the center line of the cone extends in a longitudinal direction of the cylindrical section.

5. The capsule endoscope according to claim 4, wherein the conical surface has refractive power.

6. The capsule endoscope according to claim 4, wherein the image sensor is disposed orthogonally to the optical axis of the image forming optical system, and a plane of focus of the image forming optical system is conical in shape, so that a distance between the plane of focus and the image forming optical system is longest in a vicinity of the optical axis of the image forming optical system, and is reduced with increase in a distance from the optical axis.

7. The capsule endoscope according to claim 4, further comprising:
a lighting section for applying emission light through the transparent cover to an outside of the capsule body, the lighting section being disposed in such a position that the emission light reflected from the transparent cover is not incident upon the image forming optical system.

8. The capsule endoscope according to claim 7, wherein the lighting section has a ring-shaped light emitting unit.

9. The capsule endoscope according to claim 7, wherein the lighting section has a plurality of LEDs disposed at a predetermined interval.

10. The capsule endoscope according to claim 7, wherein the lighting section is disposed on an opposite side of the image forming optical system across the reflective optical system, and a space is created between the reflective optical system and the transparent cover to pass the emission light from the lighting section therethrough.

11. The capsule endoscope according to claim 10, further comprising:
a first battery for feeding electric power to the image sensor, the first battery being disposed on a same side with the image sensor; and
a second battery for feeding electric power to the lighting section, the second battery being disposed on a same side with the lighting section.
